(19) United States
(12) Patent Application Publication
(10) Pub. No.: US 2022/0270729 A1
(43) Pub. Date: Aug. 25, 2022

(54) SYSTEMS, DEVICES, AND METHODS FOR GENERATING AND DISPLAYING MEDICAL REPORT INTERFACES AND REPORTS FOR COMMUNICATION TO A THIRD PARTY

(71) Applicant: OutcomeMD, Inc., Los Angeles, CA (US)

(72) Inventors: Justin SALIMAN, Los Angeles, CA (US); April MILLER, Los Angeles, CA (US); Jason HURST, Vancouver, WA (US); Ryan SALIMAN, Los Angeles, CA (US); Karthik KARUNANITHI, Los Angeles, CA (US); Douglas GRIM, Portland, OR (US)

(21) Appl. No.: 17/581,581

(22) Filed: Jan. 21, 2022

Related U.S. Application Data
(60) Provisional application No. 63/140,219, filed on Jan. 21, 2021.

(51) Int. Cl. G16H 15/00; G16H 10/20; G16H 10/60; H04L 9/32

(52) U.S. Cl. CPC G16H 15/00; G16H 10/20; G16H 10/60; H04L 9/3247

(57) ABSTRACT

Medical information interfaces and/or medical reports may include outcome measuring and/or tracking information such as wellness and/or improvement scores for patients who may match one or more criteria. The medical information interfaces and/or medical reports may be used for internal and/or external review and/or auditing of, for example, financial matters, resource utilization, and/or a performance (e.g., improvement scores above a threshold) of treatment providers, treatment facilities, and/or treatments for a diagnosis. The medical reports may be communicated within a closed network (e.g., within a treatment facility) and/or to a third party such as governmental agency, regulatory board, auditors, financial auditors, and/or insurance company. The medical reports and/or information included therein may be compliant with, for example, one or more formatting and/or security policies and/or protocols of, for example, requesters of the medical reports and/or a third party that may receive a medical report.

### RELATED APPLICATION

The present application is a NON-PROVISIONAL, of and claims priority to, U.S. Patent Application No. 63/140,219 filed on 21 Jan. 2021 and entitled “Systems, Devices, And Methods For Generating and Displaying Medical Report Interfaces and Reports For Communication To A Third Party,” which is incorporated in its entirety herein.

### FIELD

The present invention relates to medical information technology. More specifically, the present invention relates to systems, devices, and methods for generating and displaying medical report interfaces and reports for communication to a third party.

### BACKGROUND

Current medical and healthcare systems lack universal outcome measures and ways to objectively and scientifically track treatment outcomes for patients undergoing treatment. In addition, current medical and healthcare systems lack the ability to provide detailed reports, with treatment outcome information, of treatments provided to patients for performance, regulatory, and financial auditing.

### SUMMARY

Medical information interfaces and/or medical reports may include outcome measuring and/or tracking information such as wellness and/or improvement scores for patients who may match one or more criteria and/or are treated by a treatment provider that, in some cases, may be associated with a treatment facility. The wellness scores may be determined using one or more outcome measurement devices (e.g., medical questionnaires and/or laboratory tests) that, in some cases, may be scientifically validated (e.g., patient reported outcome instruments). When the outcome measurement device is provided in the form of a questionnaire, patients may provide responses to the questionnaire that may be scored using one or more scoring metrics and/or procedures associated with the respective questionnaire to determine a wellness score. At times, patients may respond to multiple questionnaires in order to, for example, assess various types, or areas, of wellness. For example, a patient undergoing treatment for a shoulder injury may answer medical questionnaires relating to shoulder functionality, pain management, and general health so that wellness scores for the patient's shoulder functionality, pain, and general health may be tracked over time to determine improvements (or the lack thereof) and quantify these improvements in order to, for example, measure, monitor, and/or audit treatment provider performance.

The medical information interfaces and/or medical reports disclosed herein may be used for internal and/or external review and/or auditing of, for example, financial matters, resource utilization, and/or a performance (e.g., improvement scores above a threshold) of treatment providers, treatment facilities, and/or treatments for a diagnosis. Additionally, or alternatively, The medical information interfaces and/or medical reports disclosed herein may be used to justify reimbursement from, for example, a medical insurance company or government funded program.

The medical reports may be communicated within a closed network (e.g., within a treatment facility) and/or to a third party such as governmental agency, regulatory board, auditors, financial auditors, and/or insurance company. The medical reports and/or information included therein may be compliant with, for example, one or more formatting and/or security policies and/or protocols of, for example, requesters of the medical reports and/or a third party that may receive a medical report so that, for example, information provided by the medical reports is securely provided to the third party in a format understandable by the third party's computer systems.

### BRIEF DESCRIPTION OF DRAWINGS

The present invention is illustrated by way of example, and not limitation, in the figures of the accompanying drawings in which:

FIGS. 1A and 1B are block diagrams of exemplary systems for generating and/or presenting a medical portal interface, in accordance with some embodiments of the present invention;

FIG. 2 is a block diagram showing a computer/processing system, in accordance with some embodiments of the present invention.

FIGS. 3A and 3B provide a flowchart illustrating a process for generating and providing a display of a medical report interface, in accordance with some embodiments of the present invention;

FIG. 4 provides flowchart of a process for generating a medical report, in accordance with some embodiments of the present invention;

FIG. 5A provides a screen shot of an exemplary medical information portal interface home page GUI by which information and/or criteria may be input by a user, in accordance with some embodiments of the present invention;

FIG. 5B provides a screen shot of an exemplary medical information portal interface home page GUI, in accordance with some embodiments of the present invention;

FIG. 5C provides a screen shot of an exemplary confounding injuries selection interface in accordance with some embodiments of the present invention;

FIG. 5D provides a screen shot of an exemplary medication selection interface in accordance with some embodiments of the present invention;

FIG. 5E provides an exemplary detailed data interface that provides a table that shows which treatment provider and criteria information has been received, in accordance with some embodiments of the present invention;

FIG. 5F is a screen shot of an exemplary medical report interface, in accordance with some embodiments of the present invention;

FIG. 5G is a screen shot of a report overview page GUI that includes a section for active reports and historical reports, in accordance with some embodiments of the present invention;

FIG. 5H is a screen shot of another exemplary medical report interface, in accordance with some embodiments of the present invention;

FIG. 5I is a screen shot of detailed data medical report interface, in accordance with some embodiments of the present invention; and

FIG. 5J is a screen shot of an active medical report interface that has two sections, in accordance with some embodiments of the present invention.

Throughout the drawings, the same reference numerals and characters, unless otherwise stated, are used to denote like features, elements, components, or portions of the illustrated embodiments. Moreover, while the subject invention will now be described in detail with reference to the drawings, the description is done in connection with the illustrative embodiments. It is intended that changes and modifications can be made to the described embodiments without departing from the true scope and spirit of the subject invention as defined by the appended claims.

### WRITTEN DESCRIPTION

Disclosed herein are systems, devices, and methods for generating and/or displaying medical information interfaces (e.g., graphical user interfaces (GUI)) and/or medical reports that include outcome measuring and/or tracking information such as wellness and/or improvement scores for patients who may match one or more criteria. Exemplary criteria include, but are not limited to, demographic information, diagnosis, comorbidities, confounding injuries, treatments, medication, treatment compliance information, treatment providers, treatment facilities, and/or life events associated with a patient and/or group of patients. The medical information interfaces and/or medical reports may be used for internal and/or external review and/or auditing of, for example, a performance (e.g., improvement scores above a threshold) of treatment providers, treatment facilities, and/or treatments for a diagnosis.

The medical reports may be communicated within a closed network (e.g., within a treatment facility) and/or with a third party such as governmental agency, regulatory board, performance auditors, financial auditors, and/or insurance company. The medical reports and/or information included therein may be compliant with, for example, one or more formatting and/or security policies and/or protocols of, for example, requesters of the medical reports and/or a third party that may receive a medical report.

Information displayed on the medical information interfaces and/or in the medical reports disclosed herein may include wellness scores calculated for patients (10-10,000,000) who respond to one or more medical questionnaires, such as patient reported outcome instruments that may be scientifically validated by, for example, one or more academic and/or regulatory standards. Wellness scores for individual patients may be determined over time by receiving a set of responses to the same medical questionnaires at different points in time and these separately determined wellness scores may be used to determine changes thereto over time, which may be used to determine improvement scores for the individual patient. The wellness scores and improvement scores may be aggregated, sorted, categorized and/or indexed for storage in, for example, a database that may later be queried for wellness and/or improvement scores with which to populate the medical information interfaces and/or the medical reports disclosed herein. In addition, patient characteristics may be associated with each of the wellness scores and/or improvement scores and these characterisitics may be correlated and/or indexed to the wellness and/or improvement scores in the database. Exemplary patient characterisitics include demographic information, diagnosis, treatments administered, a duration of time since treatment was administered, treatment providers, confounding injuries, comorbidities, and/or treatment facilities.

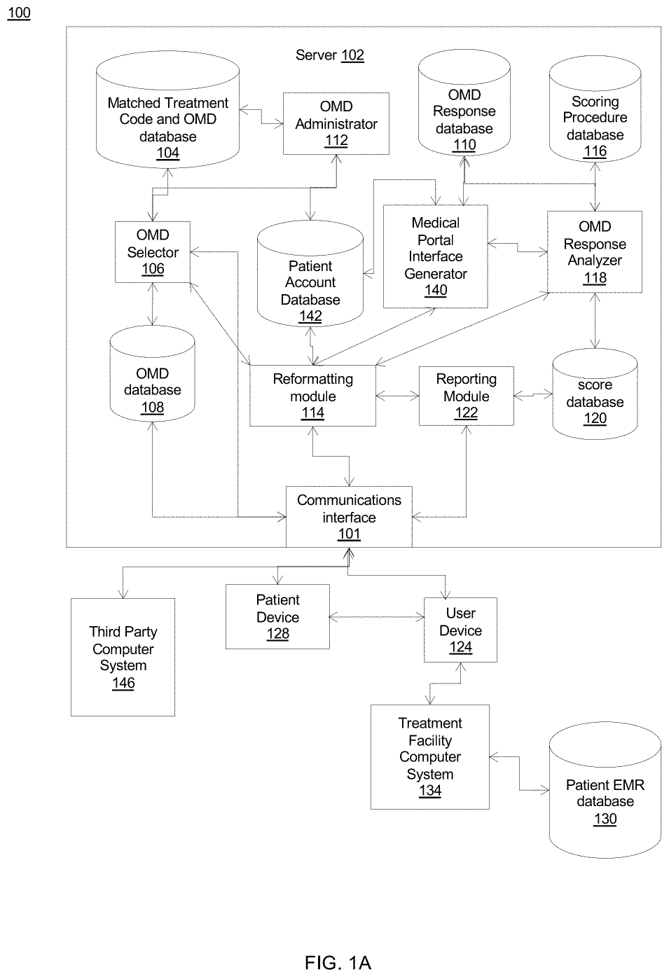

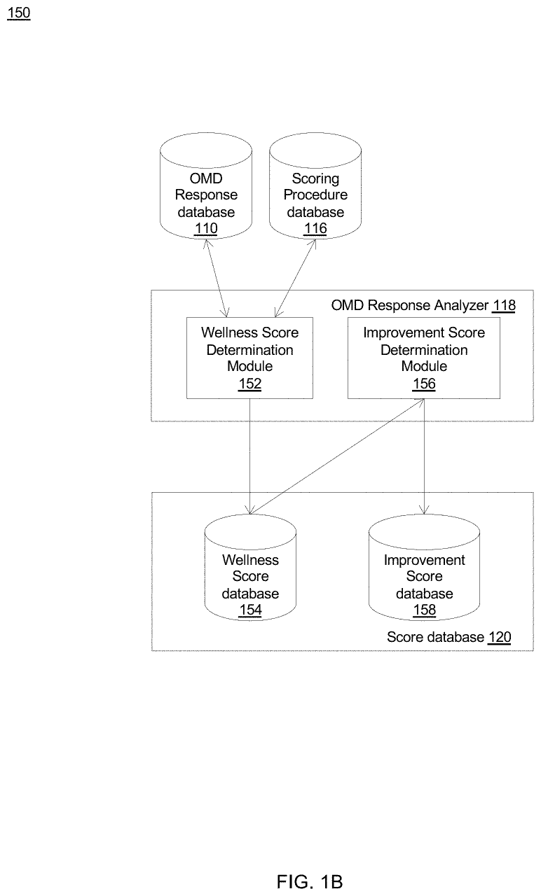

Turning now to the figures, FIG. 1A provides a block diagram of an exemplary system 100 that may be used to execute one or more of the processes described herein. At a high level, system 100 includes a server 102, a patient device 128, a user device 124, and a treatment facility computer system 134, all directly or indirectly communicatively coupled to one. Patient device 128 may be any device (e.g., a smartphone, a laptop computer, a tablet computer, a desktop computer, etc.) that enables communication between a patient and other components of system 100. Similarly, user device 124 may be any device (e.g., a smartphone, a tablet computer, a laptop computer, a desktop computer, etc.) that enables communication between a treatment provider (also referred to herein as a “user”) and other components of system 100. In some cases, user device 124 may also be a device that is enabled to perform a specific healthcare treatment and/or diagnostic task. For example, user device 124 may be a network-connected treadmill, a network connected blood pressure monitor, or a network-connected ultrasound machine. For simplicity, only one user device 124 is depicted, while it is understood that in practice there may be a plurality of user devices, one or more for each treatment provider. Similarly, while only one patient device 128 is depicted, it is understood that in practice there may be a plurality of patient devices, one or more for each patient.

Treatment facility computer system 134 may be a computer system that is located in, and/or communicatively coupled to, a treatment facility (i.e., a computer/server that is located in a doctor's office or treatment facility). As is understood in the art, an EMR (as stored in EMR database 130) may include notes prepared by a treatment provider regarding the health of a patient, results of medical tests performed on a patient, treatments administered on a patient, etc. Further due to HIPAA regulations, medical records from treatment facility computer system 134 may be communicated to user device 124, patient device 128 and server 102 using one or more security protocols that may be compliant with HIPAA requirements. It is understood that other data (i.e., not patient-specific data) may be transmitted between user device 124, patient device 128, sever 102 and facility computer system 134 via a conventional communication network (e.g., the Internet, a wired network, a wireless network, a private network, a public network, routers, switches, etc.), which has not been depicted in FIG. 1A. Further, it is understood that user device 124, patient device 128, server 102 and facility computer system 134 may be communicatively coupled to via a communication network (e.g., the Internet) and/or a blockchain.

In one embodiment, any one of the components of system 100 may replace any patient identifying information (e.g., patient name, social security number, birthdate, address, etc.) in medical records with, for example, a binary string to form anonymized medical records containing no patient identifying information (e.g., patient name, social security number, birthdate, address, etc.). More generally, any patient identifying information in medical data (e.g., EMR, questionnaire responses provided by a patient, wellness scores computed for a patient, etc.) may be replaced with a binary string to form anonymized medical data. Such anonymized medical data may be stored at, for example, server 102, treatment facility computer system 134, patient device 128, and/or user device 124, in various databases operated by server 102 (e.g., OMD response database 110, score database 120, etc.), cloud-based storage (e.g., Amazon Web services, Google Cloud platform or Microsoft Azure) (not depicted), etc. In the event the anonymized medical data is intercepted by a malicious individual (e.g., a hacker), patient privacy may still be preserved since the malicious individual will not be able to associate the anonymized medical data with any specific patient.

A mapping between respective binary strings and respective patient identifying information may be securely stored (e.g., stored in an encrypted manner) at one or more components of system 100. Such mapping may enable an electronic device (e.g., server 102, user device 124, and/or patient device 128) to access medical data associated with a specific patient. The steps for an electronic device to access the medical data of a patient may proceed as follows. First, the electronic device may be authenticated by HIPAA compliance server (e.g., the electronic device is required to provide the proper credentials, such as a login identifier and password). Following successful authentication, the electronic device may request medical data concerning an exemplary patient, John Doe. For example, server 102 may map the patient name of “John Doe” to “patient 001010” via the mapping and/or indexing, and the medical data of patient 001010 may be retrieved from a database which stores the anonymized medical data (e.g., OMD response database 110, score database 120, etc.).

In one embodiment, the process flow for system 100 may proceed as follows. Upon server 102 receiving a request from, for example, user device 124 and/or patient device 128, server 102 may provide an outcome measurement device (OMD), which may also be referred to herein as a medical questionnaire, to the patient and/or user device 124 and/or 128. An OMD may be a modality, instrument, questionnaire, or tool by which medical information about a patient may be collected. Exemplary OMDs include, but are not limited to, a medical questionnaire, a physical test of the patient (e.g., blood test, physical examination, or blood pressure), and a patient reported outcome (PRO) instrument. At times, an OMD may referred to as a medical questionnaire herein. In some cases, the request to administer the OMD may be triggered via the entry of a treatment code (e.g., a Current Procedural Terminology (CPT) code) or a treatment/diagnostic test name into the patient's EMR (as stored in patient EMR database 130), a treatment facility's billing software, and/or a treatment facility's scheduling software. In some instances, a request to administer and OMD may be triggered by a patient requesting receipt of an OMD via, for example, his or her wellness account and/or a request to administer and OMD may be triggered by a patient who requests to send an OMD to a friend or colleague via, for example, a link to an OMD and/or an invitation to respond to an OMD.

In some instances, when a treatment/diagnostic test name or other related information (other than a treatment/diagnostic code) is received, server 102 may interpret (using, for example, natural language analysis) the treatment/diagnostic test name so that it matches one or more treatment codes. In such cases, OMD selector 106 may determine one or more OMDs that match the treatment code via matched treatment code and OMD database 104. More generally, matched treatment code and OMD database 104 may also include matches between treatment names and OMDs, as well as diagnostic codes and OMDs when selecting OMDs for delivery to a patient device 128 and/or user device 124.

Next, OMD selector 106 may retrieve the one or more determined OMDs from OMD database 108. The retrieved OMDs may be provided to OMD administrator 112, which may administer the OMDs to the patient via, for example, patient device 128 and/or user device 124. In the instance that the retrieved OMDs are patient reported outcome (PRO) instruments, the PRO instruments may be provided to patient device 128. Completed OMDs (also called OMD responses) may be transmitted from patient device 128 and stored in OMD response database 110. More specifically, OMD responses may be stored in OMD response database 110 in an anonymized fashion. For example, OMD responses may be indexed in OMD response database 110 by a binary string, or other anonymous identifier, rather than by a patient name. Similarly, to the discussion above, if an OMD response for a specific patient is desired, the patient name may be mapped to a binary string by, for example, server 102, and the OMD response associated with that binary string may be retrieved from OMD response database 110.

OMD response analyzer 118 may analyze the OMD responses stored in OMD response database 110 to generate one or more scores (e.g., a wellness score, an improvement score, etc.). Such scores are described in more detail below with regard to FIG. 1B. Such analysis may rely upon scoring procedures stored in scoring procedure database 116. Such scoring may also rely upon other considerations and/or esoteric factors 132 stored at patient EMR database 130. In most circumstances, what may be referred to herein as “other considerations” are factors that may directly, or closely, relate to and/or have an impact on, a medical condition, diagnosis, and/or treatment. For example, it is known that smoking has an impact on a person's cardiovascular health. Thus, whether a person smokes may be an “other consideration” for a patient's treatment related to cardiovascular health. This relationship between cardiovascular health and smoking may be indexed or otherwise stored in other consideration database 132. An esoteric factor is one that is less directly related to a medical condition, diagnosis, and/or treatment but may still have an impact thereon. For example, a vegetarian diet may have an impact on a person's cardiovascular health, yet this impact may be less well understood when compared with the impact of smoking on the same patient's cardiovascular health. As such, a person's status as a vegetarian may be considered an “esoteric factor.”

The scores that are generated by OMD response analyzer 118 may be stored at score database 120. More specifically, scores may be stored in score database 120 in an anonymized fashion so as to, for example, comply with HIPAA regulations or other data security requirements/preferences. For instance, wellness scores associated with a patient may be indexed by a binary string in score database 120 rather than by a patient name.

Finally, reporting module 122 may report the scores to one or more of user device 124, patient device 128 and treatment facility computer system 134. In addition to the request for a treatment, there are other events that may prompt an OMD to be administered to a patient. In one example, the scheduling of an initial appointment (e.g., a consultation) for a patient to discuss a medical condition with a healthcare professional may prompt an OMD to be administered to the patient. Administering an OMD to the patient prior to this initial appointment may be useful for establishing a baseline state of health for the patient, but the selection of the OMD may have some complexity, as no treatment code, treatment name or diagnostic code may yet be available when the initial appointment is scheduled. In many instances, all that the patient will provide is a brief description of the symptoms he/she may be experiencing (e.g., shortness of breath, fever, etc.) and/or a chief complaint. In one embodiment, such symptoms may be provided to OMD selector 106, which attempts to match the symptoms with one or more treatment codes, treatment names, or diagnostic codes.

Such matching by OMD selector 106 may be performed using a learning machine. For instance, matches between, for example, symptoms and treatment codes; symptom and treatment names; and/or symptoms and diagnostic codes) may be provided by a healthcare professional when treating patients, and such matches may be used to train a model that can then be used to determine treatment codes, treatment names or diagnostic codes based on, for example, a patient's symptoms and/or treatment provider notes. Upon determining a treatment code, treatment name, or a diagnostic code, OMD selector 106 may select one or more OMDs based on matches provided in matched treatment code and OMD database 104 (as described above). It is anticipated that the determination of a treatment code, treatment name or diagnostic code by OMD selector 106 may be, in some instances, an imperfect process, so a healthcare provider, or other expert, may be asked to make any necessary adjustments to the treatment code, treatment name and/or diagnostic code determination, before OMD selector 106 selects the one or more OMDs.

In the examples provided above, it was assumed that an OMD is administered to a patient via patient device 128. In other instances, a medical professional may be required to administer the OMD to the patient. For example, server 102 may notify user device 124 that one or more OMDs should be administered as part of, for example, a medical examination of a patient. In one example, if a patient has recently undergone cardiothoracic surgery, OMD administrator 112 may provide one or more OMDs to user device 124 (e.g., the Intrathoracic Gas Volume Test, Total Lung Capacity Test, Vital Capacity Test, 6 Minute Walk Test, Aortic Insufficiency Test, Mitral Regurgitation Test and/or Aortic Valve Area Test) that could, or should, be administered to the patient during an exam and/or provide one or more mechanisms to user device 124 (e.g., fillable forms) for the treatment provider to enter the OMD responses.

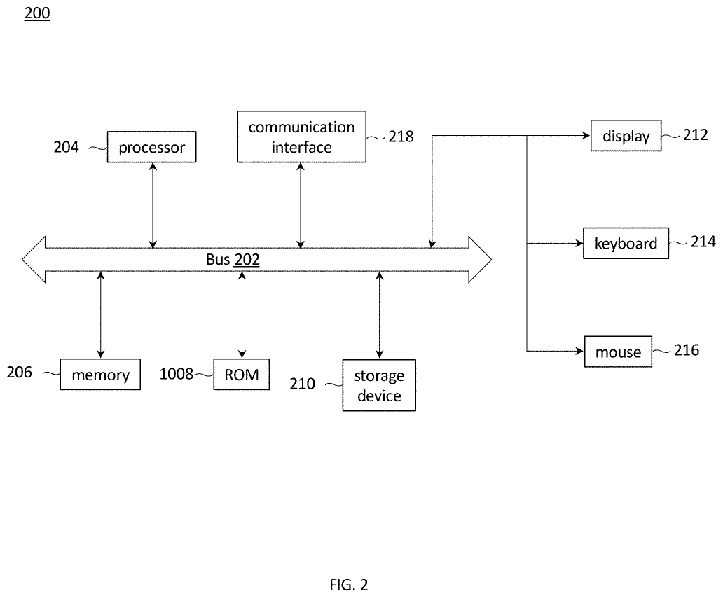

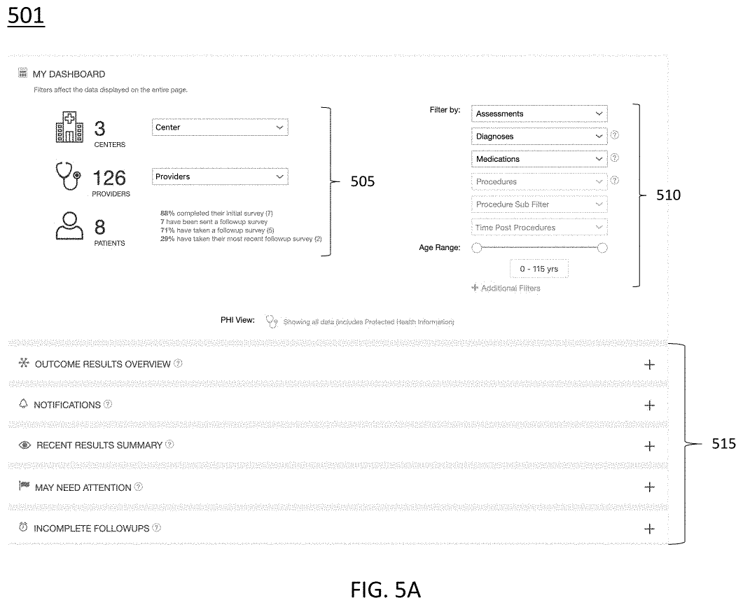

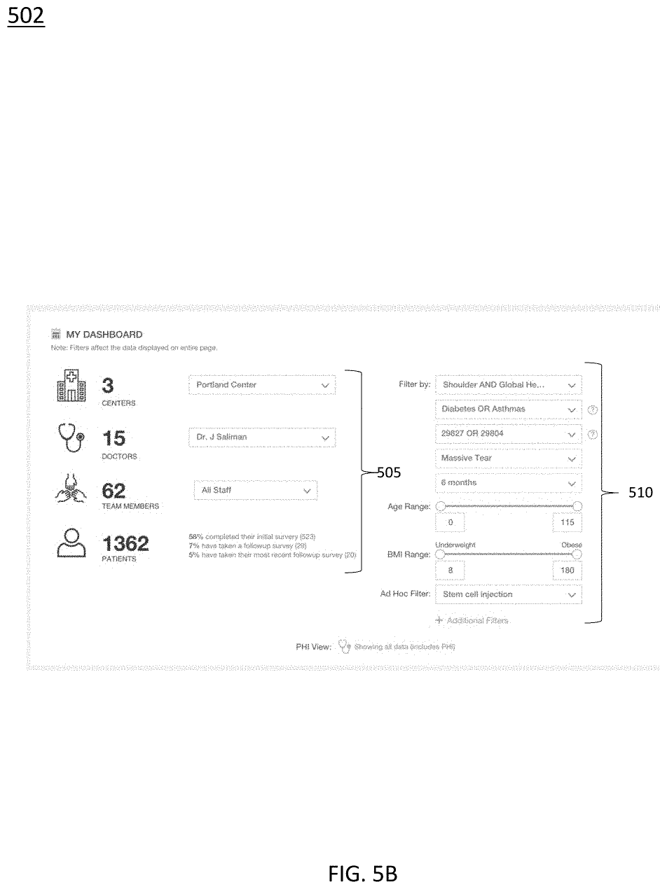

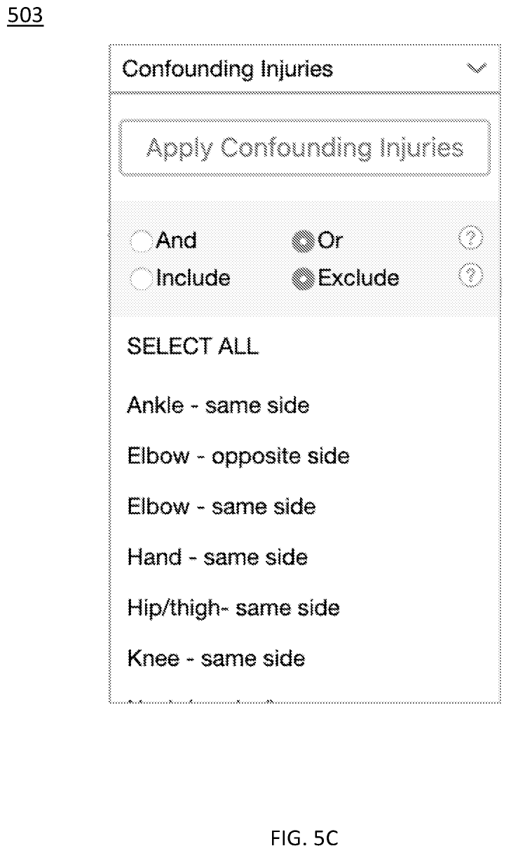

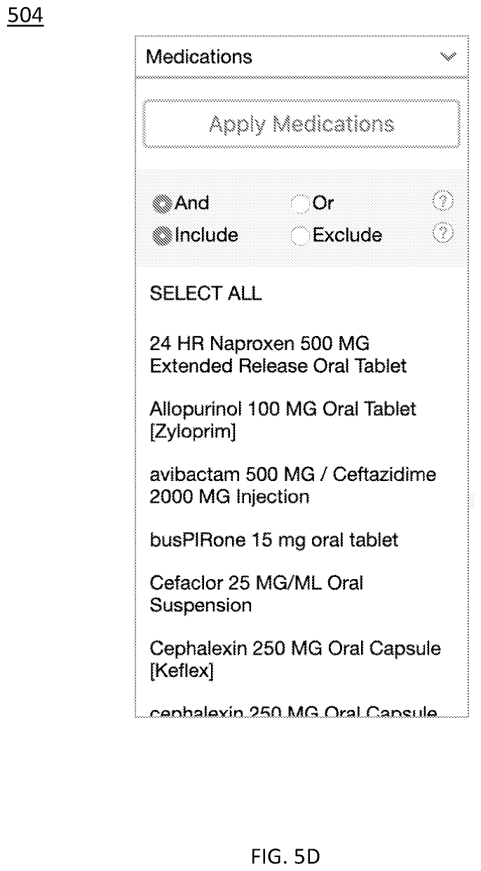

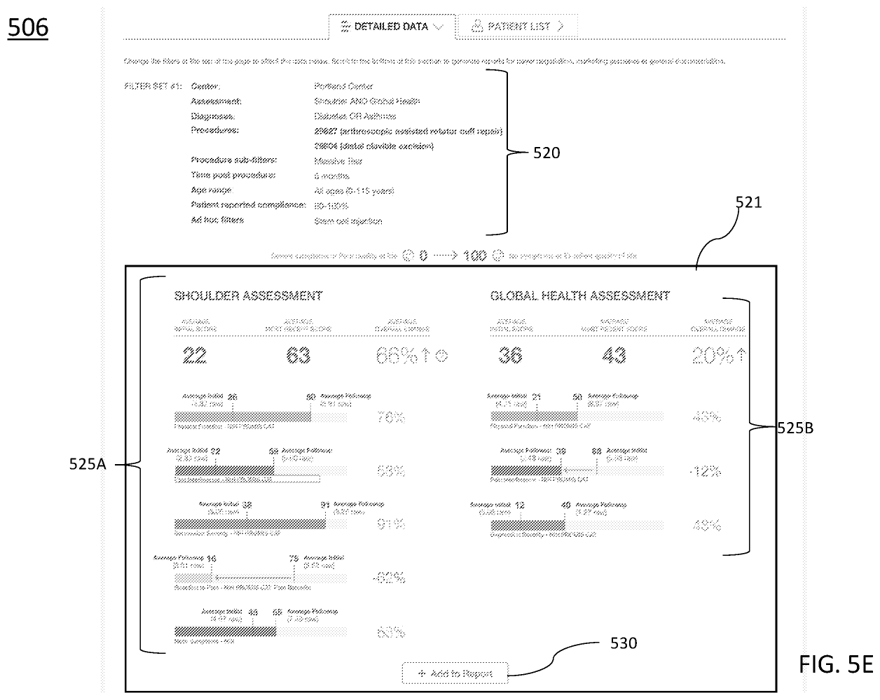

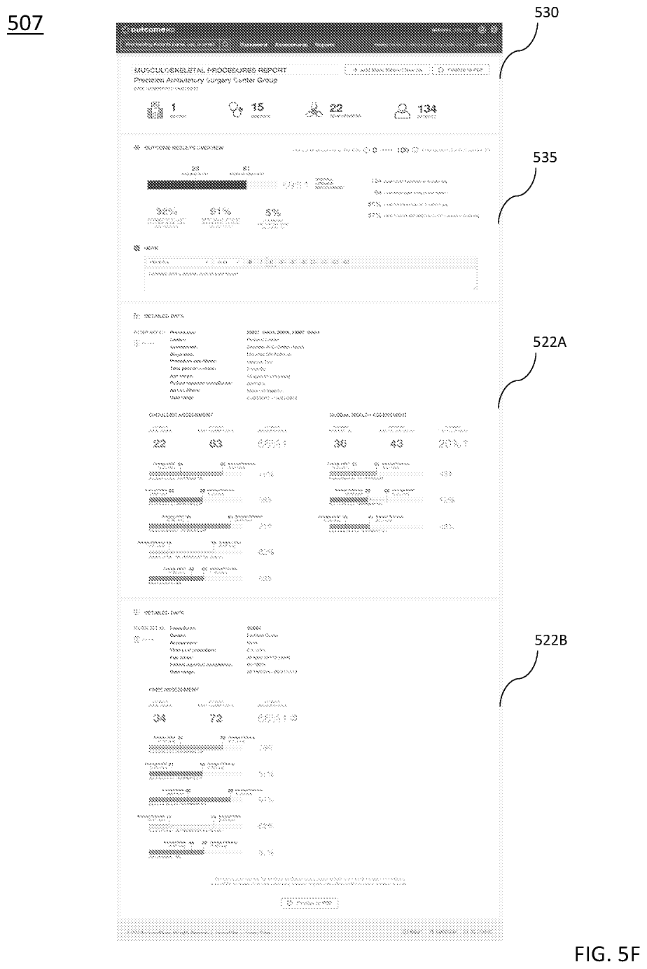

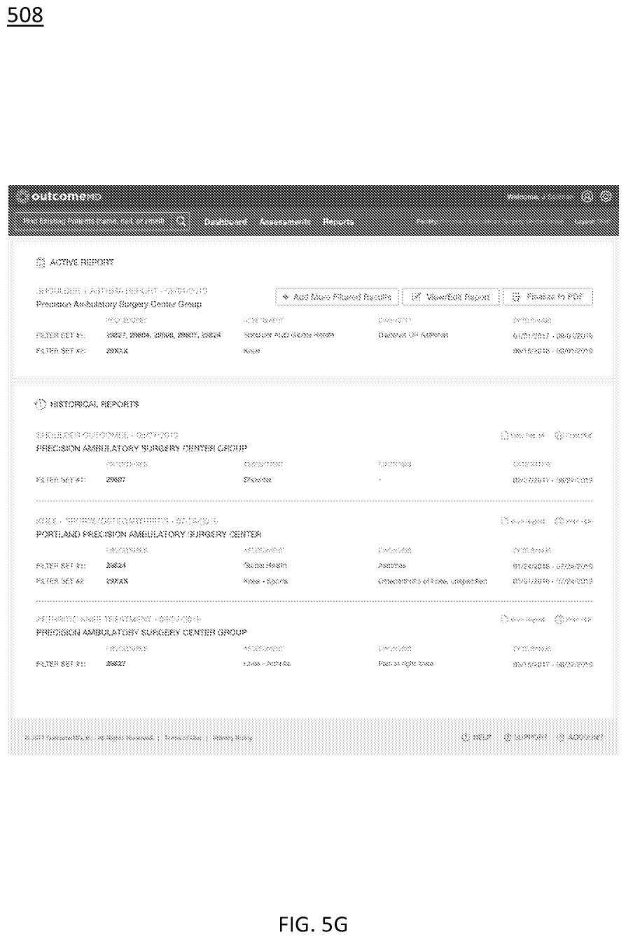

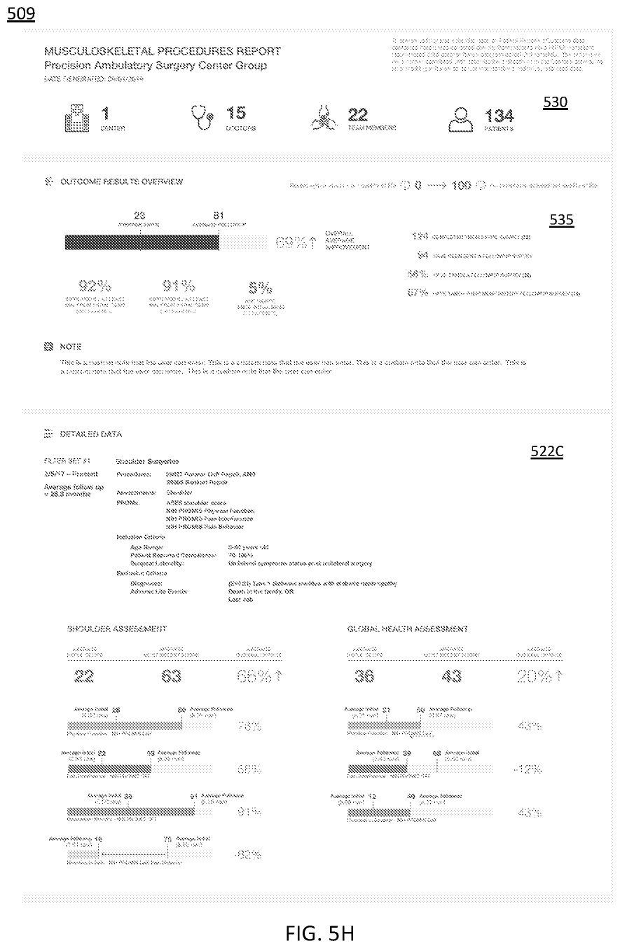

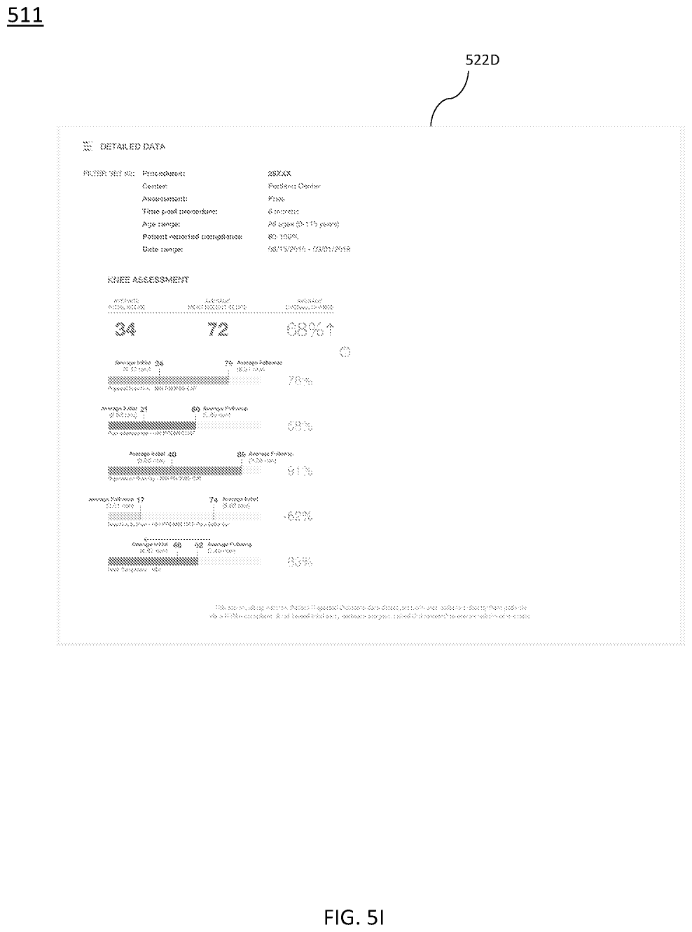

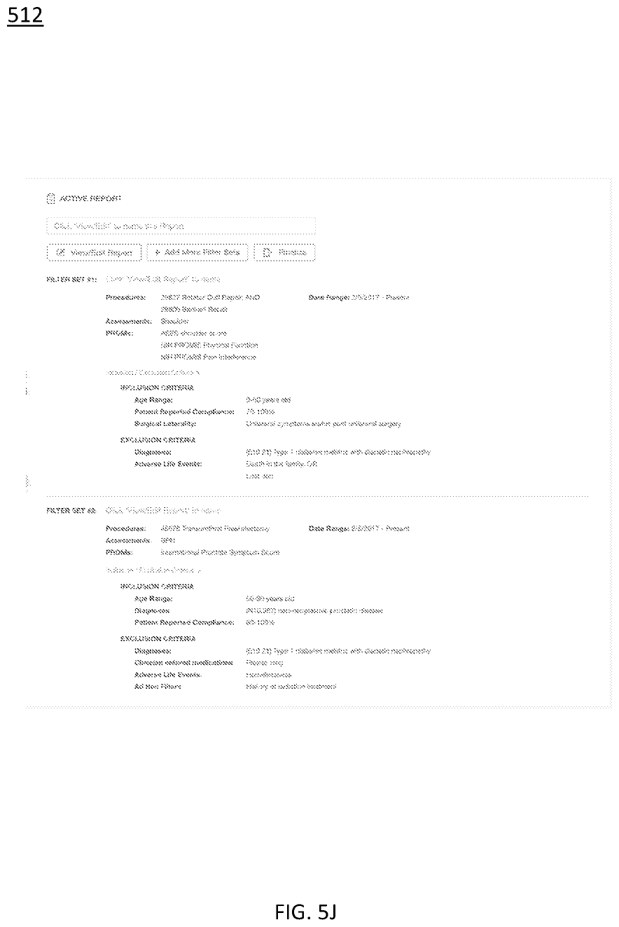

Server 102 may further include a patient account database 142, a medical portal interface generator 140, a reformatting module 114, and a communications interface 101. Communication interface 101 may facilitate communication between server 102 and an external device such as third-party computer system 146, patient device 128, and/or user device 124. In some embodiments, communication interface 101 may resemble communication interface 1118. Medical portal interface generator 140 may generate one or more medical portal interfaces, like the medical portal interfaces shown in FIGS. 2A-C and FIGS. 5A-10B, using information received by and/or stored in server 102. For example, medical portal interface generator 140 may receive information from a user, patient, and/or third-party information source via, for example, user device 124, patient device 128, and/or third-party computer system 146. The received information may be reformatted from an original and/or intermediate format into a format compatible with, for example, server 102 and/or medical portal interface generator 140 by reformatting module 114. The received information may then be added to and/or used to generate a medical portal interface by medical portal interface generator 140. Additionally, or alternatively, medical portal interface generator 140 may receive information from patient account database 142 (e.g., in response to a query sent by the medical portal interface generator 140 to patient account database 142) that may be used to add to and/or generate a medical portal interface by medical portal interface generator 140. Exemplary information that may be received from patient account database 142 includes, but is not limited to, patient identifiers, demographic information for one or more patients, patient-entered information (e.g., adverse life events, medication/treatment compliance, answers to OMDs, supplemental treatments, etc.), treatment history, historical OMD responses, wellness scores, improvements scores, and so on.

Third-party computer system 146 may be computer system operated by an entity that is not the patient and/or operated by and/or directly associated with a user of user device 124 (e.g., treatment facility staff or medical professionals) and/or an entity operating server 102. Examples include pharmacies, governmental agencies (e.g., Center for Medicare and Medicaid Services (CMS), Food and Drug Administration (FDA) and/or local medical boards), regulatory bodies (e.g., American Medical Association) medical treatment facilities other than medical treatment facilities coupled to treatment facility computer system 134 and/or patient EMR database 130 (e.g., laboratories, radiologists, chiropractors, physical fitness facilities, etc.), medical device retailers, and other service providers for patients such as ride-share services that may be able to provide information regarding pick-up and drop-off times for patients at a facility that may administer medical treatment.

In some cases, medical portal interface generator may pull together information from various third-party information sources to build a complete picture of the health and/or treatment of one or more patients so that it may be conveyed via a medical portal interface like the medical portal interfaces of FIGS. 2A-2C and 5A-10B for analysis and study by a user.

Patient accounts may be associated with each individual patient under the care of a particular treatment facility. Information about a patient may be associated with and/or stored along with patient account information. Information about a patient may come from a plurality of sources including, but not limited to, the patient, a treatment provider, a user of a server providing access to a patient account, and a third-party.

Patient accounts may be generated at/by server 102 responsively to instructions from the patient (as provided via, for example, patient device 128) and/or responsively to a user like a treatment facility administrator or medical treatment provider providing instructions via, for example, user device 124. Often times, the patient account is not directly linked (e.g., can receive information from and/or provide information to) all medical treatment and/or care providers. The medical treatment and/or care providers not in direct communication with a patient account

FIG. 1B depicts one embodiment of a system 150 that supports the operation of OMD response analyzer 118 and score database 120 (and some associated components). OMD response analyzer 118 may comprise wellness score determination module 152. In one embodiment, wellness score determination module 152 retrieves responses to an OMD from OMD response database 110, and further may retrieve a scoring procedure associated with the OMD responses from scoring procedure database 116. The scoring procedures may be indexed by, for example, an identifier of an OMD, for which responses have been received, making for easy retrieval of a corresponding scoring procedure. Various scoring procedures may be employed to score a completed OMD, and in one embodiment, the generated score may be known as a “wellness score”. In some cases, a “wellness score” may serve to indicate how severe a patient's symptoms are. In these cases, a low wellness score may indicate that a patient's symptoms are relatively more severe than a higher wellness score such that a subsequent higher wellness score indicates an improvement (i.e., decrease in severity) in the symptoms.

In the case where an OMD is a questionnaire (or PRO instrument), a certain weighing may be used to score or evaluate the patient's responses. For example, certain responses that are more objective in nature (e.g., heart rate, blood glucose level, etc.), may receive greater weights (and hence have a greater influence on the wellness score) than certain responses that are more subjective in nature (e.g., degree of pain, mood, etc.). The reverse scenario, of course, could be true in which subjective responses receive a greater weight than objective responses (e.g., fatigue or mental illness). Scores generated by wellness score determination module 152 may be stored in wellness score database 154. The wellness scores may be indexed in various fashions, for ease of retrieval. In one embodiment, wellness scores may be indexed according to one or more of a patient identifier (e.g., binary string to protect patient privacy), medical condition, treatment provider, treatment facility, time at which OMD was completed, etc.

Improvement score determination module 156 may retrieve two wellness scores for a patient (e.g., a first score calculated for an OMD completed at a first time point and a second score calculated for an OMD completed at a second time point) from wellness score database 154. Improvement score determination module 156 may calculate the difference between the first and second score, and such difference may be known as an improvement score. The improvement score may be stored in improvement score database 158. In one refinement, a relative improvement score may be calculated as the improvement score (i.e., the difference described above) normalized by a maximum improvement score, which may be calculated based on, for example, other considerations 132 stored in a patient's EMR. The maximum improvement score may take into consideration other factors such as the state of a patient prior to a medical treatment (e.g., if patient was in fairly good health, the maximum improvement score might be lower than if the patient was in poor health), and/or the age of a patient (e.g., younger patients might have a higher maximum improvement score than older patients), etc. An improvement score (or a relative improvement score) may be stored in improvement score database 158. The improvement scores may be indexed in various fashions, for ease of retrieval. In one embodiment, improvement scores may be indexed according to one or more of a patient identifier (e.g., binary string to protect patient privacy), medical condition, treatment provider, treatment facility, and time duration over which improvement score was measured, etc.

The components and/or databases of systems 100, 150, and/or 103 of FIGS. 1A, 1B, and/or 1C may be a series of one or more components (e.g., computers, servers, databases, etc.) that may, in some instances, be geographically disparate.

As disclosed herein, a wellness score and/or an improvement score for one or more aspects of the patient's medical condition and/or physiological systems may then be determined by scoring responses to one or more assessments, which in some cases may be patient reported outcome (PRO) assessments that have been validated to assess a patient's medical condition via medical literature and/or accepted best practices within the medical field. In some embodiments, determination of a wellness score may include querying a scoring database like scoring procedure database 116, for a scoring metric and/or scoring procedure associated with the medical questionnaire provided in step 205. In some instances, this querying may include retrieving a scoring procedure from scoring procedure database 116 using an identifier of the medical questionnaire. For instance, a medical questionnaire may be associated with a code (e.g., 3232) and this code may be used to retrieve a scoring procedure from scoring procedure database 116. Example scoring procedures include taking an average of all the patient responses (e.g., assuming all responses are numeric), taking a weighted average of the patient responses (e.g., weighting certain responses higher than other responses), adjusting the range of patient responses (e.g., changing responses choices from 2, 3, 3 to 1, 5, 6). In some embodiments, determining a wellness score may include retrieval of a sub-scoring procedure that may be specific to the patient (i.e., associated with the patient's account and/or a co-morbidity of the patient) as may be indicated by, for example, the patient's account and/or EMR. The scored responses may then be used to determine a wellness score associated with the received responses and/or a sub-set of received responses.

An improvement score (or percentage change) may be a determination of how a patient's condition has changed over time. In some embodiments, determination of an improvement score may involve comparing (e.g., averaging, subtracting, determining a percentage change, determining a time weighted average, etc.) one or more previously determined wellness scores with a currently determined wellness score in order to determine how a patient's wellness score has changed over time (e.g., 3 weeks, 3 months, 1 year, etc.).

FIG. 2 is a block diagram showing a system 200 includes a bus 202 or other communication mechanism for communicating information, and a processor 204 coupled with the bus 202 for processing information. Computer system 200 also includes a main memory 206, such as a random-access memory (RAM) or other dynamic storage device, coupled to the bus 202 for storing information and instructions to be executed by processor 204. Main memory 206 also may be used for storing temporary variables or other intermediate information during execution of instructions to be executed by processor 204. Computer system 200 further includes a read only memory (ROM) 208 or other static storage device coupled to the bus 202 for storing static information and instructions for the processor 204. A storage device 22, for example a hard disk, flash memory-based storage medium, or other storage medium from which processor 204 can read, is provided and coupled to the bus 202 for storing information and instructions (e.g., operating systems, applications programs and the like).

Computer system 200 may be coupled via the bus 202 to a display 212, such as a flat panel display, for displaying information to a computer user. An input device 214, such as a keyboard including alphanumeric and other keys, may be coupled to the bus 202 for communicating information and command selections to the processor 204. Another type of user input device is cursor control device 216, such as a mouse, a track pad, or similar input device for communicating direction information and command selections to processor 204 and for controlling cursor movement on the display 212. Other user interface devices, such as microphones, speakers, etc. are not shown in detail but may be involved with the receipt of user input and/or presentation of output.

The processes referred to herein may be implemented by processor 204 executing appropriate sequences of computer-readable instructions contained in main memory 206. Such instructions may be read into main memory 206 from another computer-readable medium, such as storage device 22, and execution of the sequences of instructions contained in the main memory 206 causes the processor 204 to perform the associated actions. In alternative embodiments, hard-wired circuitry or firmware-controlled processing units may be used in place of or in combination with processor 204 and its associated computer software instructions to implement the invention. The computer-readable instructions may be rendered in any computer language.

In general, all of the above process descriptions are meant to encompass any series of logical steps performed in a sequence to accomplish a given purpose, which is the hallmark of any computer-executable application. Unless specifically stated otherwise, it should be appreciated that throughout the description of the present invention, use of terms such as “processing”, “computing”, “calculating”, “determining”, “displaying”, “receiving”, “transmitting” or the like, refer to the action and processes of an appropriately programmed computer system, such as computer system 200 or similar electronic computing device, that manipulates and transforms data represented as physical (electronic) quantities within its registers and memories into other data similarly represented as physical quantities within its memories or registers or other such information storage, transmission or display devices.

Computer system 200 also includes a communication interface 218 coupled to the bus 202. Communication interface 218 may provide a two-way data communication channel with a computer network, which provides connectivity to and among the various computer systems discussed above. For example, communication interface 218 may be a local area network (LAN) card to provide a data communication connection to a compatible LAN, which itself is communicatively coupled to the Internet through one or more Internet service provider networks. The precise details of such communication paths are not critical to the present invention. What is important is that computer system 200 can send and receive messages and data through the communication interface 218 and in that way communicate with hosts accessible via the Internet. It is noted that the components of system 200 may be located in a single device or located in a plurality of physically and/or geographically distributed devices.

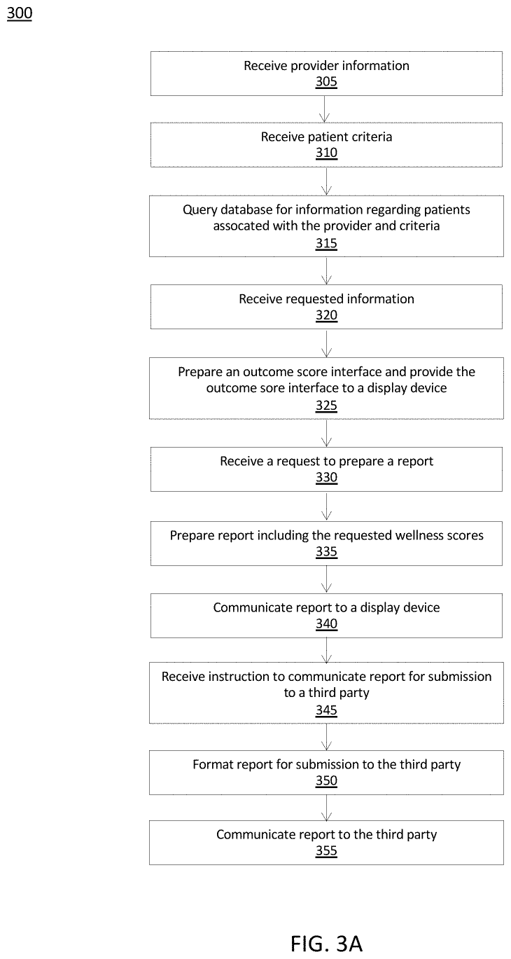

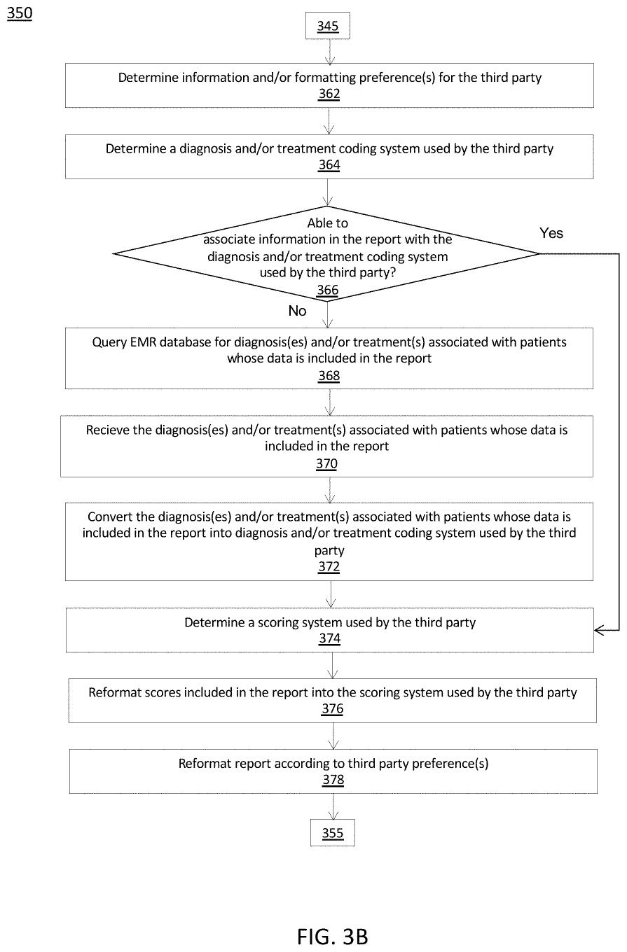

FIG. 3 provides a flowchart illustrating a process 300 for generating a medical report interface. Process 300 may be executed by, for example, systems 100, 101, and/or 200 and/or components thereof.

In step 305, treatment provider information may be received. Treatment provider information may include information regarding any provider of medical and/or wellness treatment including, but not limited to, treatment centers, doctors, groups of doctors, teams, team members, dieticians, coaches, counsellors, and/or other medical professionals (e.g., nurses, lab technicians, etc.).

In step 310, patient criteria may be received. Patient criteria may include any criteria by which a patient and/or information about a patient may be selected. Exemplary patient criteria include, but are not limited to, an OMD, medical questionnaire, lab test, and/or assessment the patient has completed, a diagnosis, medications prescribed, medical procedures performed, sub-filters for medical procedures, a duration of time following a procedure, an age range, a confounding factor, a weight range, a body mass index, supplemental treatments, adverse life events, a time period for treatment administration, and/or an indicator of patient compliance with a treatment program or the taking of medication. In some instances, the criteria may be exclusionary. For example, if a patient is not compliant with a treatment regimen, the patient may be excluded from a report.

In some cases, multiple criteria (e.g., a first, second, and third criteria) may be received in step 310. At times, some of the criteria (e.g., the first and second criterion) may be used to query a database to extract a set of patients who have a particular diagnosis A and a particular treatment B. The third criteria may be an exclusionary factor, or filter, that may be applied to a data set of patients matching diagnosis A and treatment B but have a confounding factor that would adversely interfere with healing from treatment B and/or improvement of diagnosis A. For example, if the criteria received were a diagnosis of a rotator cuff tear where the patient completed the shoulder assessment and the treatment procedures were arthroscopic assisted rotator cuff repair and a distal clavicle excision, this set of criteria may be used to query a database to extract a set of patients who meet all of these criteria. A confounding factor (e.g., an arm injury or broken elbow) may then be applied to this set of patients in order to, for example, remove outliers from the patient set who have a confounding injury that may interfere with recovery from the treatment.

FIG. 5A provides a screen shot of an exemplary medical information portal interface home page GUI 501 by which the information and/or criteria received in step(s) 305 and/or 310 may be input by a user. Medical information portal interface home page GUI 501 provides a provider menu 505 by which a user may enter information regarding a provider of medical treatment. More specifically, provider menu 505 provides a drop-down menu by which a user may select a medical facility or medical center (referred to on provider menu 505 as a “centers”) and drop-down menu by which a user may select a provider, which may be a single treatment provider or individual or group of providers/individuals. Provider menu 505 may also include a list of patients and/or information about those patients who are receiving treatment from a selected center and/or provider.

Medical information portal interface home page GUI 501 also provides a diagnosis/treatment menu 510 by which a user may select one or more diagnosis, treatment, confounding factors, and/or filters to apply to patient data prior to displaying it on a report. Exemplary categories of information that may be entered by a user via diagnosis/treatment menu 510 include assessments, ages, diagnoses, procedures, medications, procedure sub filters, time post procedure, follow up ranges, and a date range. Diagnosis/treatment menu 510 also provides an “+additional filters” icon; the selection of which may cause interface 501 to provide a number of additional filters the user may apply to the data. Additional filters include, but are not limited to, confounding injuries (which may be general (e.g., any confounding injury) or specific (e.g. specific to a diagnosis or treatment)) body mass index (BMI), and ad hoc filters which may be manually entered by a user and/or selected via a drop down menu.

In addition, medical information portal interface home page GUI 501 provides a list of tabs 515, the selection of which may cause display of an associated interface, page, GUI, and/or window. Tabs provided in list of tabs 515 include an outcome results overview tab, a notification tab, a recent results summary tab, a may need attention tab, and an incomplete follow-up tab. Filters and/or categories selected from first and/or second-layer menus 505 and/or 510 may be applied to information displayed on one or more medical report interfaces and/or the pages and/or GUIs accessed via selection of a corresponding tab from list 515.

FIG. 5B provides a screen shot of an exemplary medical information portal interface home page GUI 502 that displays the information received in step(s) 305 and/or 310. For example, provider menu 505 shows that the user has input “Portland Center” as the treatment center, “Dr. J. Saliman” as the treatment provider or doctor, and “all staff” as the team members as the provider information into interface 601/402 and that these inputs were received in step 305. Diagnosis/treatment menu 510 of FIG. 5B shows that the user has input patient criteria in the form of an assessment of “Shoulder AND Global He . . . ”, which stands for global health assessment, a confounding factor of “diabetes OR Asthmas,” a procedure code of “29827 OR 29804,” a diagnosis of “massive tear,” and an ad hoc filter of “stem cell injection” into interface 501/502, which were received in step 310.

FIG. 5C provides a screen shot of an exemplary confounding injuries selection interface 503 that provides a user with a plurality of possible confounding injuries for patients that the user may select as criteria, which may be received as patient criteria in step 310.

In some embodiments, confounding injuries may be associated with one or more patients upon receipt of an indication that the patient is suffering from a confounding injury, or a problem that may be limiting the patient's activity when the patient answers questions of a medical questionnaire like the medical questionnaires used to populate a database like database OMD response database 110. In one example, if the patient who is under treatment for a left side shoulder injury responds to a question of a medical questionnaire by stating that he or she is suffering from a musculoskeletal confounding injury, the patient may be asked one or more follow up question(s) regarding the confounding injury. For example, this patient may be asked to select one or more regions (e.g., upper extremity, lower extremity, thorax, head, neck, etc.) of the patient's body which are causing problems or pain. If the patient selects, for example, upper extremity, the patient may be asked whether the injury/problem pertains to:

Shoulder—opposite side

Shoulder—same side

Elbow—opposite side

Elbow—same side

Hand/wrist—opposite side

Hand/wrist—same side

via an interface like confounding injuries selection interface 503.

FIG. 5D provides a screen shot of an exemplary medication selection interface 504 that provides a user with a plurality of possible medications that may be prescribed to patients that the user may select as criteria, which may be received as patient criteria in step 310. The process for associating patient questionnaire responses with one or more of the medications listed in interface 504 may be similar to the process for associating patients with confounding injuries discussed above.

In step 315, one or more databases, like OMD response database 110, patient account database 142, patient EMR database 130, score database 120, wellness score database 154, and/or improvement score database 158 may be queried to extract a set of patients who are associated with the provider and the patient criteria. The information requested may be, for example, wellness scores, improvement scores, and/or changes in wellness scores pre and/or post treatment and/or changes in wellness scores over time. In step 320, the requested information may be received from the database. Then, an outcome score interface may then be prepared using the received information and provided to a display device (step 325). Then a request to prepare a report using the received information and/or other information included in the outcome score interface may be received (step 330). The requested report may be prepared (step 335) and the prepared report may be communicated to a display device like display 212 (step 340).

FIG. 5E provides an exemplary detailed data interface 506 that provides a table 520 that shows which treatment provider and criteria information has been received in step 305 and/or 310. Detailed data interface 506 also shows an outcome window 521 that includes two sets of outcome data 525 with a first set of outcome data 525A showing outcome data for a shoulder assessment and a second set of outcome data 525B showing outcome data for a global health assessment for the patients who took the shoulder assessment.

In addition, detailed data interface 506 also provides an add to report button 530 which when selected by a user communicates an instruction to a processor to format the information provided in and/or by detailed data interface 506 into a report format for use by, for example, the user. An example of a report generated responsively to receipt of a user's activation and/or selection of add to report button is provided by the screen shots of FIGS. 5G-5J.

The first set of outcome data 525A shows, among other things, an average initial score of 22 for shoulder assessments, an average most recent score of 63, and an average overall change of an increase of wellness scores by 66%. The same patient population is also associated with global health assessment scores of 36 for the initial average initial score, 63 for the average most recent score, and an average overall change of an increase of wellness scores by 20% as shown in second set of data 525B for a global health assessment.

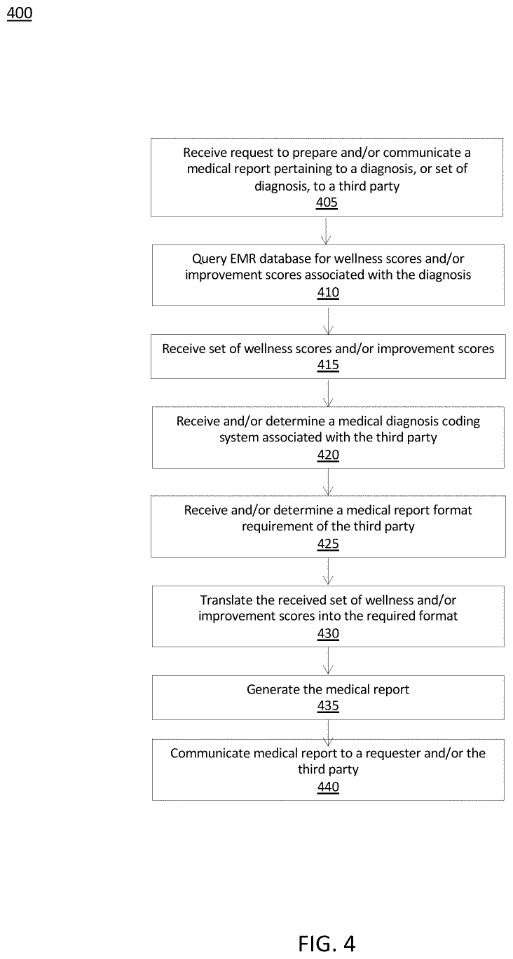

Optionally, an instruction to communicate the report to a third party such as an insurance company, medical review board, governmental agency, or other third party may be received (step 345). The report may then be formatted (step 350) in a manner compatible with the third party (e.g., placed in a text-only format (e.g., a rich text format (RTF)), in a comma separated value format (CSV), and/or PDF format). In some cases, execution of step 350 may include de-identifying the information presented in the report so that it removes personal information for individual patients. Once the report is formatted, it may be communicated to the third party (step 355). Further details regarding formatting the report are provided herein with regard to FIG. 4 and process 400.

In some embodiments, execution of step 350 may include associating one or more aspects of a report such as wellness scores, improvement scores, and/or compliance scores with, for example, a diagnosis and/or treatment code (e.g., CPT codes or ICD-10 codes) that is compatible with a database and/or software operated by the recipient third party. Further details regarding how step 350 may be executed are provided in FIG. 3B, which depicts a process for reformatting the report in a manner compatible with the third party and also provided in FIG. 4 and discussed below. For example with regard to FIG., in step 362, information regarding formatting preferences for the third party may be determined and, in step 364, a diagnosis and/or treatment coding system used by the third party (e.g., a proprietary set of codes, CPT codes, and/or ICD-10 codes) to understand, for example, patient diagnosis and/or treatments provided to patients that are covered by a report may be determined. In some embodiments, the information and/or formatting preferences for the third party may include an accounting or other software program that the third party uses to, for example, manage its internal operations and/or issue payments to healthcare providers.

In step 366, it may be determined whether information in and/or associated with the report is in a format compatible with the recipient/third party and, if so, process 350 may proceed to step 374. More specifically, in step 366, it may be determined whether information in and/or associated with the report is compatible with a diagnosis and/or treatment coding (e.g., CPT codes or IDC-10 codes) system, outcome reporting system, and/or accounting system used by the third party. When not compatible, an EMR database like patient EMR database 130 may be queried for diagnosis(es) and/or treatments associated with the patients whose data is included in the report (step 368). Then the requested diagnosis(es) and/or treatments associated with the patients whose data is included in the report may be received (step 370) and converted into the diagnosis and/or treatment coding system used by the third party. Additionally, or alternatively, treatment provider database may be queried for information needed to converting outcome reporting and/or accounting information into a format compatible with the third party's data format and/or systems.

Optionally, in step 374, a scoring system (e.g., wellness scores, improvement scores, etc.) used by the third party may be determined. When the scoring system is not compatible with the third party, the scores associated with the report may be reformatted to be compatible with the third party (step 376) and then the report may be reformatted or converted into a format compatible with third party preferences (step 378) and process 300 may proceed to step 355, described above.

FIG. 5F is a screen shot of an exemplary medical report interface 507 that may be generated in step 335, communicated to the display device in step 340, and displayed on the display device. Medical report interface 507 includes a first detailed data window 522A that provides data that is similar to the detailed data shown in interface 506 of FIG. 5E and a second detailed data window 522B that provides detailed information regarding knee assessments performed at, or otherwise associated with, the Portland Center. Medical report interface 507 also includes a heading 530 and an outcome heath overview window 535. Heading 530 includes general information about the report and who it pertains to and outcome heath overview window 535 provides overview information regarding all of the patients treated by, or otherwise associated with, the treatment facility (in this case, the Precision Ambulatory Surgery Center Group) such as overall average improvement percentages and the number of patients treated by the treatment facility. Heath overview window 535 also provides a note section in which a user may add a customized note to the report.

FIG. 5G is a screen shot of a medical report overview GUI 508 that includes a section for active reports and historical reports. The active reports section provides information regarding the title of the report and filters used to generate the report. It also provides a series of user-selectable user interface buttons, the selection of which may enable a user to add more filtered results, view/edit an active report, and/or finalize a report to a PDF or other format.

FIG. 4 provides a flowchart illustrating a process 400 for generating a medical report and communicating same to a requester and/or third party. Process 400 may be executed by, for example, systems 100, 101, and/or 200 and/or components thereof.

Initially, a request to communicate a medical report corresponding to a medical diagnosis, treatment provider, and/or treatment facility to a third party may be received by, for example, server 102, patient device 128, user device 124, and/or treatment facility computer system 134 (step 405) via, for example, a GUI like the medical report interface and/or home page GUIs disclosed herein. The third party may be, for example, a third party associated with third party computer system 146. The medical report may include a plurality of outcome-based wellness scores determined for a plurality of patients diagnosed with the medical diagnosis and/or treated by the treatment provider and/or treatment facility. The wellness scores may be determined by, for example, scoring responses from one or more patient reported outcome questionnaires received from each patient of the plurality of patients. The medical report may also include improvement scores that may, for example, indicate changes in the wellness scores for each respective patient of the plurality of patients over time as disclosed herein.

In step 410, an electronic medical record database such as, for example, patient EMR database 130, may be queried for wellness scores and improvement scores associated with the diagnosis, treatment provider, and/or treatment facility and a set of wellness scores and improvement scores associated with the diagnosis, treatment provider, and/or treatment facility may be received from the electronic medical record database responsively to the query (step 415). One or more aspects, or features, of providing reports to the third party may then be determined and/or received responsively to, for example, a query of the third party computer system. For example, a medical diagnosis coding system used by the third party and/or a medical diagnosis code of the medical treatment coding system that is correlated to the medical diagnosis may be received and/or determined (step 420) and/or a medical report formatting requirement of the third party may be received and/or determined (step 425).

In step 430, the set of the wellness scores and/or improvement scores received in step 415 may be translated into a format compliant with the medical report formatting requirement of the third party using a determined medical report formatting requirement of the third party received and/or determined in step 425. A medical report using a translated set of the wellness scores and improvement scores may then be generated (step 435) and provided to, for example, the requester and/or the third party (step 440). An exemplary medical report that may be generated via execution of process 400 is provided in FIGS. 5G, 5H, 5I, and 5J.

In some embodiments, the formatting requirement of the third party received and/or determined in step 425 may be a requirement for a format for use when auditing a treatment provider and/or treatment facility providing a treatment for the diagnosis and the medical report may also include financial information and execution of step 430 and/or 435 may include translating the medical report information into an accounting format compatible with the third party's accounting system. Additionally, or alternatively, the formatting requirement of the third party may be a requirement for a format for use when determining a treatment outcome-based performance for a treatment provider providing a treatment for the diagnosis as may be indicated by, for example, comparing improvement scores proved by the medical report with a threshold improvement score value by, for example, the third party.

In another example, the formatting requirement of the third party may be a scale (e.g., 1-5, 1-100, and/or A-F) for the determination and/or reporting of wellness scores and/or improvement scores and execution of step 430 may include normalizing or otherwise adapting the scale of the wellness scores to align and/or comply with the third party's scale for the determination of wellness scores.

Additionally, or alternatively, the formatting requirement of the third party may be use of an encryption and/or cryptographic protocol (e.g., public/private key encryption, transport layer security (TLS) encryption, and/or secure socket layer security (SSL) encryption, encryption for use on a blockchain) to encrypt, digitally lock, digitally sign, and/or digitally certify a medical report received by the third party and execution of step 430 may include encrypting the medical report with the encryption protocol prior to providing the medical report report to the requester and/or communicating the medical report using a communication protocol required by the third party. Additionally, or alternatively, the formatting requirement of the third party may be that the medical report be digitally signed with a digital security certificate and/or digitally locked to prevent modification and execution of step 430 may include digitally signing and/or digitally locking the medical report with a digital security certificate the medical report prior to providing the medical report report to the requester.

Additionally, or alternatively, the formatting requirement of the third party may require that the medical report be communicated via and/or stored on a blockchain. In these instances, execution of step 430 may include packaging the medical report to be stored on the blockchain prior to providing the medical report report to the requester and broadcasting a packaged medical report to the blockchain. At times, the medical report may be subject to a smart contract and communication of the medical report to the third party and/or broadcasting the medical report to a blockchain may fulfill the smart contract. For example, payment of invoices from a treatment provider may be part of a smart contract wherein once the medical reports are communicated to the third party, payment of the invoice is initiated by the third party.

In some embodiments, the request received in step 405 may include one or more characterisitics and/or factors by which to, for example, search for, filter, and/or sort information included in the medical report and these filtration and/or sorting requests may be included in, for example, the EMR query of step 410 so that the set of wellness scores and improvement scores associated with the diagnosis, treatment provider, and/or treatment facility are filtered and/or sorted to remove patients receiving the treatment for the diagnosis who are diagnosed with the comorbidity prior to generating the medical report report. Exemplary characterisitics and/or factors by which to search for, filter, and/or sort information included in the medical report include, but are not limited to, comorbidities, confounding factors, demographic information, diagnosis, and/or treatments administered to the patient(s).

FIGS. 5H-5J provide screen shots of another set of exemplary medical report interfaces 509, 511, and 512, respectively, that may be generated using one or more processes described herein. More specifically, FIG. 5H is a screen shot of another exemplary medical report interface 509 that may be generated and communicated to a display device in step(s) 340 and/or 355, so that the report may be displayed on the display device. Medical report interface 509 includes heading 530, outcome heath overview window 535, and a first detailed data window 522C. Information displayed in an upper portion of first detailed data window 522C provides details regarding filters that have been set for inclusion and exclusion criteria for the report. More specifically, the data displayed on exemplary medical report interfaces 509 is subject to the following filters:

General Category: shoulder surgeries

Time period: Feb. 5, 2015-present

Procedures: 29827 Rotator Cuff Repair and 29806 Bankart Repair

Assessments taken by patients:
Shoulder
ASES shoulder score
NIH PROMIS Physical Function
NIH PROMIS Pain Interference
NIH PROMIS Pain Behavior
   The data is also subject to inclusion and exclusion criteria with the inclusion criteria being:

Age range: 0-60 years old

Patient reported compliance: 70-100%

Surgical laterality: Unilateral symptoms status post unilateral surgery and exclusion criteria being:

Diagnosis: (E10.21) Type 1 diabetes mellitus with diabetic neuropathy

Adverse life events: death in the family or lost job.

These filters are then applied to the available data and the results are provided in third detailed data window 522C as well as fourth detailed data window 522D as shown in detailed data medical report interface 511 of FIG. 5I.

FIG. 5J provides a screen shot of an active medical report interface 512 that has two sections with a first section providing information regarding filter set #1 as shown on third detailed window 522C and the second section providing information regarding filter set #1 as shown on fourth detailed window 522D. By providing the ability to set both inclusion and exclusion criteria for the patients selected for inclusion in a report, a user may be able to extract clinically significant data regarding patients associated with a plurality of characteristics, treatments, outcomes, and so on from a variety of sources such as OMD response database, patient account database 142, score database 120, and/or patient EMR database 130, reformat the data from these varying sources so that it is compatible with one another so that a centralized report may be generated, and provide that report to the user. In some cases, the report, once the generated and/or once the data used to generate a report is gathered, may formatted so that it is compatible with the software and/or systems of a third party who is, for example, evaluating, auditing, and/or paying a treatment provider.

We claim:

1. A computer-implemented method, comprising:
receiving a request to communicate a medical report corresponding to a medical diagnosis to a third party, the medical report including outcome-based wellness scores determined for a plurality of patients diagnosed with the medical diagnosis using scored responses from respective each patient of the plurality of patients for one or more patient reported outcome questionnaires and improvement scores that indicate changes in the wellness scores for each respective patient of the plurality of patients over time;
querying an electronic medical record database for wellness scores and improvement scores associated with the diagnosis;
receiving a set of wellness scores and improvement scores associated with the diagnosis from the electronic medical record database;
determining a medical diagnosis coding system used by the third party and a medical diagnosis code of the medical treatment coding system correlated to the medical diagnosis;
determining an medical report formatting requirement of the third party;
translating the set of the wellness scores and improvement scores into a format compliant with the medical report formatting requirement of the third party using a determined medical report formatting requirement of the third party;
generating an medical report using a translated set of the wellness scores and improvement scores; and
providing the medical report report to the requester.

2. The computer-implemented method of claim 1, wherein the formatting requirement of the third party is a format for use when auditing a treatment provider providing a treatment for the diagnosis.

3. The computer-implemented method of claim 1, wherein the formatting requirement of the third party is a format for use when determining a treatment outcome-based performance for a treatment provider providing a treatment for the diagnosis.

4. The computer-implemented method of claim 1, wherein the formatting requirement of the third party is a scale for the determination of wellness scores and the translating the set of wellness scores into a format compliant with the medical report formatting requirement of the third party includes normalizing the scale of the wellness scores to the third party's scale for the determination of wellness scores.

5. The computer-implemented method of claim 1, wherein the formatting requirement of the third party is a scale for the determination of improvement scores and the translating the set of improvement scores into a format compliant with the medical report formatting requirement of the third party includes normalizing the scale of the improvement scores to the third party's scale for the determination of improvement scores.

6. The computer-implemented method of claim 1, wherein the formatting requirement of the third party is an encryption protocol, the method further comprising:
encrypting the medical report with the encryption protocol prior to providing the medical report report to the requester.

7. The computer-implemented method of claim 1, wherein the formatting requirement of the third party is that the medical report be digitally signed with a digital security certificate, the method further comprising:
digitally signing the medical report with a digital security certificate the medical report prior to providing the medical report report to the requester.

8. The computer-implemented method of claim 1, wherein the formatting requirement of the third party is that the medical report be digitally locked to prevent modification prior to communication to the third party, the method further comprising:
digitally locking the medical report prior to providing the medical report report to the requester.

9. The computer-implemented method of claim 1, wherein the formatting requirement of the third party requires the medical report be stored on a blockchain, the method further comprising:
packaging the medical report to be stored on the blockchain prior to providing the medical report report to the requester; and
broadcasting a packaged medical report to the blockchain.

10. The computer-implemented method of claim 9, wherein provision of the packaged medical report to the third party fulfils a requirement of a smart contract.

11. The computer-implemented method of claim 1, further comprising:
providing the medical report report directly to the third party.

12. The computer-implemented method of claim 1, further comprising:
receiving a request to filter patients receiving a treatment for the diagnosis who are diagnosed with a comorbidity; and
filtering the set of wellness scores and improvement scores associated with the diagnosis to remove patients receiving the treatment for the diagnosis who are diagnosed with the comorbidity prior to generating the medical report report.

13. The computer-implemented method of claim 1, further comprising:
receiving a request to filter patients receiving a treatment for the diagnosis who are diagnosed with a confounding injury; and
filtering the set of wellness scores and improvement scores associated with the diagnosis to remove patients receiving the treatment for the diagnosis who are diagnosed with the confounding injury prior to generating the medical report report.

14. The computer-implemented method of claim 1, wherein the plurality of patients are treated for the diagnosis by a treatment facility.

15. A computer-implemented method, comprising:
receiving a request to communicate a medical diagnosis and treatment report corresponding to a treatment provider to a third party, the medical report including outcome-based wellness scores determined for a plurality of patients treated by the treatment provider using scored responses from respective each patient of the plurality of patients for one or more patient reported outcome questionnaires and improvement scores that indicate changes in the wellness scores for each respective patient of the plurality of patients over time;
querying an electronic medical record database for wellness scores and improvement scores associated with the treatment provider;
receiving a set of wellness scores and improvement scores associated with the treatment provider from the electronic medical record database;
determining an medical report formatting requirement of the third party;
translating the set of the wellness scores and improvement scores into a format compliant with the medical report formatting requirement of the third party using a determined medical report formatting requirement of the third party;
generating an medical report report using a translated set of the wellness scores and improvement scores; and
providing the medical report report to the requester.

16. The computer-implemented method of claim 15, wherein the formatting requirement of the third party is a format for use when auditing the treatment provider.

17. The computer-implemented method of claim 15, wherein the formatting requirement of the third party is that the medical report be digitally signed with a digital security certificate, the method further comprising:
digitally signing the medical report with a digital security certificate the medical report prior to providing the medical report report to the requester.

18. The computer-implemented method of claim 15, wherein the formatting requirement of the third party is that the medical report be digitally locked to prevent modification prior to communication to the third party, the method further comprising:
digitally locking the medical report prior to providing the medical report report to the requester.

19. The computer-implemented method of claim 15, wherein the formatting requirement of the third party requires the medical report be stored on a blockchain, the method further comprising:
packaging the medical report to be stored on the blockchain prior to providing the medical report report to the requester; and
broadcasting a packaged medical report to the blockchain.

20. The computer-implemented method of claim 19, wherein provision of the packaged medical report to the third party fulfils a requirement of a smart contract.

21. The computer-implemented method of claim 15, further comprising:
receiving a request to filter patients receiving a treatment for the diagnosis who are diagnosed with a comorbidity; and
filtering the set of wellness scores and improvement scores associated with the diagnosis to remove patients receiving the treatment for the diagnosis who are diagnosed with the comorbidity prior to generating the medical report report.

22. The computer-implemented method of claim 15, further comprising:
receiving a request to filter patients receiving a treatment for the diagnosis who are diagnosed with a confounding injury; and
filtering the set of wellness scores and improvement scores associated with the diagnosis to remove patients receiving the treatment for the diagnosis who are diagnosed with the confounding injury prior to generating the medical report report.
